SEQUENCE LISTING (61 sequences)

SEQ ID NO: 1 - protein, 21 aa, Artificial Sequence
  Description of Artificial Sequence Synthetic probe
KLVFFAEDVGSNKGAIIGLMK

SEQ ID NO: 2 - protein, 21 aa, Artificial Sequence
  Description of Artificial Sequence Synthetic probe
KLVFFAEDVGSNKHAIIELMK

SEQ ID NO: 3 - protein, 21 aa, Artificial Sequence
  Description of Artificial Sequence Synthetic probe
KLVFFAEDAAAAKHAIIELMK

SEQ ID NO: 4 - protein, 25 aa, Artificial Sequence
  Description of Artificial Sequence Synthetic probe
KAAAKLVFFAEDVGSNKHAIIELMK

SEQ ID NO: 5 - protein, 21 aa, Artificial Sequence
  Description of Artificial Sequence Synthetic probe
KLVFFAEDVGSNKGAISGLMK

SEQ ID NO: 6 - protein, 21 aa, Artificial Sequence
  Description of Artificial Sequence Synthetic probe
KLVFFAEDVGSNKGAIIGLAK

SEQ ID NO: 7 - protein, 21 aa, Artificial Sequence
  Description of Artificial Sequence Synthetic probe
KLVFFAPDVGSNKGAIIGLMK

SEQ ID NO: 8 - protein, 21 aa, Artificial Sequence
  Description of Artificial Sequence Synthetic probe
KLVSFAEDVGSNKGAIIGPMK

SEQ ID NO: 9 - protein, 21 aa, Artificial Sequence
  Description of Artificial Sequence Synthetic probe
KLVFFGEDVGSNKGAIIGLMK

SEQ ID NO: 10 - protein, 21 aa, Artificial Sequence
  Description of Artificial Sequence Synthetic probe
KLVFFAGDVGSNKGAIIGLMK

SEQ ID NO: 11 - protein, 21 aa, Artificial Sequence
  Description of Artificial Sequence Synthetic probe
KLVFFAQDVGSNKGAIIGLMK

SEQ ID NO: 12 - protein, 21 aa, Artificial Sequence
  Description of Artificial Sequence Synthetic probe
KLVFFAKDVGSNKGAIIGLMK

SEQ ID NO: 13 - protein, 21 aa, Artificial Sequence
  Description of Artificial Sequence Synthetic probe
KLVFFAENVGSNKGAIIGLMK

SEQ ID NO: 14 - protein, 21 aa, Artificial Sequence
  Description of Artificial Sequence Synthetic probe
KLVFFAEDVGSNKRAIIELMK

SEQ ID NO: 15 - protein, 21 aa, Artificial Sequence
  Description of Artificial Sequence Synthetic probe
KLVFFAEDVGSNKKAIIELMK

SEQ ID NO: 16 - protein, 26 aa, Artificial Sequence
  Description of Artificial Sequence Synthetic probe
HHQKLVFFAEDEGSRKHAIEGLMEGK

SEQ ID NO: 17 - protein, 26 aa, Artificial Sequence
  Description of Artificial Sequence Synthetic probe
EAAKLVFFAEDEGSRKHAIEGLMEGK

SEQ ID NO: 18 - protein, 21 aa, Artificial Sequence
  Description of Artificial Sequence Synthetic probe
KLVFFAEDVGSNKGAISGLMK

SEQ ID NO: 19 - protein, 21 aa, Artificial Sequence
  Description of Artificial Sequence Synthetic probe
KLVFFAKDVGSNKHAIIELMK

SEQ ID NO: 20 - protein, 21 aa, Artificial Sequence
  Description of Artificial Sequence Synthetic probe
KLVFFAQDVGSNKHAIIELMK

SEQ ID NO: 21 - protein, 21 aa, Artificial Sequence
  Description of Artificial Sequence Synthetic probe
KLVFFAGDVGSNKHAIIELMK

SEQ ID NO: 22 - protein, 23 aa, Artificial Sequence
  Description of Artificial Sequence Synthetic probe
KLVFFAEDVGSNKGAIIGLMKRR

SEQ ID NO: 23 - protein, 23 aa, Artificial Sequence
  Description of Artificial Sequence Synthetic probe
KLVFFAEDVGSNKHAIIELMKRR

SEQ ID NO: 24 - protein, 25 aa, Artificial Sequence
  Description of Artificial Sequence Synthetic probe
RRKLVFFAEDVGSNKHAIIELMKEE

SEQ ID NO: 25 - protein, 5 aa, Artificial Sequence
  Description of Artificial Sequence Synthetic peptide
GXXEG

SEQ ID NO: 26 - protein, 27 aa, Artificial Sequence
  Description of Artificial Sequence Synthetic probe
KAAAKLVFFAEDVGSNKHAIIELMKRR

SEQ ID NO: 27 - protein, 27 aa, Artificial Sequence
  Description of Artificial Sequence Synthetic probe
KAAAKLVFFAEDVGSNKGAIIGLMKRR

SEQ ID NO: 28 - protein, 21 aa, Artificial Sequence
  Description of Artificial Sequence Synthetic probe
KLVFFAEDVGSNKDADIELMK

SEQ ID NO: 29 - protein, 21 aa, Artificial Sequence
  Description of Artificial Sequence Synthetic probe
KLVFFAEDVGDNKHAIIELMK

SEQ ID NO: 30 - protein, 21 aa, Artificial Sequence
  Description of Artificial Sequence Synthetic probe
KLVFFAEDVGSNKHADIELMK

SEQ ID NO: 31 - protein, 21 aa, Artificial Sequence
  Description of Artificial Sequence Synthetic probe
KLVFFAEDVGSNKHAIIEDMK

SEQ ID NO: 32 - protein, 21 aa, Artificial Sequence
  Description of Artificial Sequence Synthetic probe
KLVFFAEDVGSNKDAIIELMK

SEQ ID NO: 33 - protein, 21 aa, Artificial Sequence
  Description of Artificial Sequence Synthetic probe
KLVFFAEDVGDNKHADIELMK

SEQ ID NO: 34 - protein, 21 aa, Artificial Sequence
  Description of Artificial Sequence Synthetic probe
KLVSFAEDVGSNKHAIIEPMK

SEQ ID NO: 35 - protein, 21 aa, Artificial Sequence
  Description of Artificial Sequence Synthetic probe
KLVSFAEDVGSNKHAIIELMK

SEQ ID NO: 36 - protein, 21 aa, Artificial Sequence
  Description of Artificial Sequence Synthetic probe
KLVFFAEDVGSNKGAIIGLMK

SEQ ID NO: 37 - protein, 25 aa, Artificial Sequence
  Description of Artificial Sequence Synthetic probe
EKLVFFAEDVGSNKGAIIGLMKRRR

SEQ ID NO: 38 - protein, 26 aa, Artificial Sequence
  Description of Artificial Sequence Synthetic probe
KLVFFAEDVGSNKHAIIELMKEAAAK

SEQ ID NO: 39 - protein, 21 aa, Artificial Sequence
  Description of Artificial Sequence Synthetic probe
KLVFFAEDVGSNKHAIIELMK

SEQ ID NO: 40 - protein, 28 aa, Artificial Sequence
  Description of Artificial Sequence Synthetic probe
KLVFFAEDVGSNKHAIIELMKGSSGSSK

SEQ ID NO: 41 - protein, 30 aa, Artificial Sequence
  Description of Artificial Sequence Synthetic probe
KLVFFAEDVGSNKHAIIELMKGLVPRGSGK

SEQ ID NO: 42 - protein, 27 aa, Artificial Sequence
  Description of Artificial Sequence Synthetic probe
KLVFFAEDVGSNKHAIIELMKPSGSPK

SEQ ID NO: 43 - protein, 21 aa, Artificial Sequence
  Description of Artificial Sequence Synthetic probe
KLVFFAEDVGSNKHAIIELMK

SEQ ID NO: 44 - protein, 21 aa, Artificial Sequence
  Description of Artificial Sequence Synthetic probe
ELVFFAEDVGSNKGAIIGLMK

SEQ ID NO: 45 - protein, 40 aa, Artificial Sequence
  Description of Artificial Sequence Synthetic probe
ERHDSGYEVHHQKLVFFAEDVGSNKGAIIGLMVGGVVIAK

SEQ ID NO: 46 - protein, 35 aa, Artificial Sequence
  Description of Artificial Sequence Synthetic probe
EEFRHDSGYEVHHQKLVFFAEDVGSNKGAIIGLMK

SEQ ID NO: 47 - protein, 27 aa, Artificial Sequence
  Description of Artificial Sequence Synthetic probe
EAAAKLVFFAEDEGSRKHAIEGLMEGK

SEQ ID NO: 48 - protein, 27 aa, Artificial Sequence
  Description of Artificial Sequence Synthetic probe
EHHQKLVFFAEDEGSRKHAIEGLMEGK

SEQ ID NO: 49 - protein, 21 aa, Artificial Sequence
  Description of Artificial Sequence Synthetic probe
ELVFFAEDVGSNKHAIIELMK

SEQ ID NO: 50 - protein, 21 aa, Artificial Sequence
  Description of Artificial Sequence Synthetic probe
KLVFFAEDVGSNKHAIIELMW

SEQ ID NO: 51 - protein, 21 aa, Artificial Sequence
  Description of Artificial Sequence Synthetic probe
KLVFFAEDVGSNKGAIIGLME

SEQ ID NO: 52 - protein, 21 aa, Artificial Sequence
  Description of Artificial Sequence Synthetic probe
KLVFFAEDVGSNKHAIIELME

SEQ ID NO: 53 - protein, 34 aa, Artificial Sequence
  Description of Artificial Sequence Synthetic probe
EFRHDSGYEVHHQKLVFFAEDVGSNKGAIIGLME

SEQ ID NO: 54 - protein, 26 aa, Artificial Sequence
  Description of Artificial Sequence Synthetic probe
HHQKLVFFAEDEGSRKHAIEGLMEGE

SEQ ID NO: 55 - protein, 26 aa, Artificial Sequence
  Description of Artificial Sequence Synthetic probe
EAAKLVFFAEDEGSRKHAIEGLMEGE

SEQ ID NO: 56 - protein, 21 aa, Artificial Sequence
  Description of Artificial Sequence Synthetic probe
KLVFFAKDVGSNKGAIIGLMK

SEQ ID NO: 57 - protein, 5 aa, Artificial Sequence
  Description of Artificial Sequence Synthetic peptide
EAAAK

SEQ ID NO: 58 - protein, 7 aa, Artificial Sequence
  Description of Artificial Sequence Synthetic peptide
GSSGSSK

SEQ ID NO: 59 - protein, 6 aa, Artificial Sequence
  Description of Artificial Sequence Synthetic peptide
LVPRGS

SEQ ID NO: 60 - protein, 9 aa, Artificial Sequence
  Description of Artificial Sequence Synthetic peptide
GLVPRGSGK

SEQ ID NO: 61 - protein, 6 aa, Artificial Sequence
  Description of Artificial Sequence Synthetic peptide
PSGSPK

(19) United States
(12) Patent Application Publication
(10) Pub. No.: US 2012/0282169 A1
(43) Pub. Date: Nov. 8, 2012

(54) DETECTION AND TREATMENT OF TRAUMATIC BRAIN INJURY

(75) Inventors: D. Roxanne Duan, Bethesda, MD (US); Jonathan R. Moll, Rockville, MD (US); Alan Rudolph, Potomac, MD (US)

(73) Assignee: Adlyfe, Inc.

(21) Appl. No.: 13/508,212

(22) PCT Filed: Nov. 4, 2010
(86) PCT No.: PCT/US2010/055429

§ 371 Date: Jul. 23, 2012

Related U.S. Application Data
(60) Provisional application No. 61/258,837, filed on Nov. 6, 2009.

(51) Int. Cl. G01N 21/64; A61K 51/00; A61K 49/06; A61K 49/00

(52) U.S. Cl. 424/1.11; 436/501; 435/7.8; 424/9.6; 424/9.3; 424/9.5; 424/9.1

(57) ABSTRACT

The present invention relates to the detection of traumatic brain injury by detecting Aβ protein aggregates associated with traumatic brain injury. These Aβ protein aggregates are detected using peptide and peptide mimic probes that preferentially associate with Aβ protein aggregates associated with traumatic brain injury.

### GOVERNMENT SUPPORT

This invention was made with United States government support under Contract No. W911NF09C0087 awarded by the Defense Advanced Research Projects Agency and the U.S. Army Research Office. The United States government has certain rights in the invention.

### BACKGROUND

1. Field of the Invention

The present invention relates to the field of the detection of proteins associated with traumatic brain injury. More particularly, the present invention relates to methods for detecting amyloid-β (Aβ) protein aggregates associated with traumatic brain injury, in vivo or in vitro.

2. Background

Studies have demonstrated a link between traumatic brain injury (TBI) and the amyloid events associated with protein folding neurodegenerative brain diseases. These include deleterious accumulation of amyloid proteins and associated pathology in Alzheimer's Disease, Parkinson's Disease, vascular dementia and others. Human epidemiology studies and amyloid mutant transgenic mouse studies have shown that repetitive or even single incident brain trauma increases susceptibility to developing neurodegenerative amyloid disease including Alzheimer's Disease (AD) (Chen, X. H. C. et al., Journal of Neurotrauma 2004, 21, (9), 1291-1291; Uryu, K. et al., Experimental Neurology 2007, 208, (2), 185-192). In fact, TBI is the most robust environmental AD risk factor (Guo, Z. et al., Neurology 2000, 54, (6), 1316-1323; Heyman, et al., Annals of Neurology 1984, 15, (4). 335-341; Mortimer, J. et al., Neurology 1985, 35, (2), 264-267; Plassman, B. L. et al., Neurology 2000, 55, (8), 1158-1166). For example, soldiers are at high risk for brain trauma due to blast injury or other direct CNS trauma, with associated damage of soft and hard tissue. If the brain trauma is diagnosed early, TBI victims could be treated to prevent progressive brain amyloidosis and the onset of AD, hence dramatically improving health and reducing long-term care expenses. There is a need, therefore, for methods for the detection and diagnosis of TBI.

### SUMMARY OF THE INVENTION

The present invention provides a method for detecting Aβ protein aggregates associated with traumatic brain injury in a physiological sample from a subject, comprising: (A) contacting the sample with a peptide or peptide mimic probe, wherein the probe (i) preferentially associates with the Aβ protein aggregates, (ii) undergoes a conformation shift upon association with the Aβ protein aggregates, and (iii) generates a detectable signal when the probe associates with the Aβ protein aggregates; and (B) detecting any association between the probe and any Aβ protein aggregate present in the sample.

In one embodiment, the probe is labeled with a detectable label that generates a signal when the probe associates with the Aβ protein aggregates. In a further embodiment, the probe is labeled at separate sites with a first label and a second label, generating a signal when the probe undergoes a conformation shift upon association with Aβ protein aggregates. In a further embodiment, the sites of the first and second label are selected from (i) the N-terminus and the C-terminus; (ii) the N-terminus and a separate position other than the C-terminus; (iii) the C-terminus and a separate position other than the N-terminus; and (iv) two positions other than the N-terminus and the C-terminus.

In one embodiment, first and second labels are excimer-forming labels. In a further embodiment, the first and second labels comprise pyrene or a fluorophore/quencher pair. In an alternative embodiment, the first label comprises one member of a fluorescent resonance energy transfer (FRET) pair and the second label comprises the other member of the FRET pair.

In another embodiment, the conformation shift is selected from the group consisting of (a) adopting a conformation upon association with the Aβ protein aggregate that increases the physical proximity of the first and second labels; and (b) adopting a conformation upon association with the Aβ protein aggregate that decreases the physical proximity of the first and second labels.

Physiological samples used in the method may be selected from brain tissue, cerebrospinal fluid, whole blood, serum, plasma, eye tissue, vascular tissue, lung tissue, kidney tissue, heart tissue and liver tissue.

In one embodiment of the invention, the probe is a peptide probe. In a further embodiment, the peptide probe consists of from 10 to 50 amino acid residues corresponding to a β-sheet forming region of Aβ protein, wherein the amino acid sequence of the probe is at least 60%, at least 75%, at least 80%, at least 85%, at least 90%, at least 95%, or 100% identical to the corresponding region of Aβ protein. In an alternative embodiment of the invention, the probe is a peptide or peptoid mimic.

In one embodiment of the invention, the traumatic brain injury is due to physical or chemical trauma. In a further embodiment, the traumatic brain injury is selected from the group consisting of closed head injury, penetrating head injury, focal brain injury, diffuse brain injury, concussion, dementia pugilistica, anesthesia-related injury, isoflurane-related injury and shaken baby syndrome.

The present invention also provides an in vivo method for detecting Aβ protein aggregates associated with traumatic brain injury, comprising: (A) administering to the patient a peptide or peptide mimic probe, wherein the probe (i) preferentially associates with the Aβ protein aggregate, (ii) undergoes a confoi illation shift upon association with the Aβ protein aggregate, and (iii) is labeled with a detectable label that generates a signal when the probe associates with the Aβ protein aggregates; and (B) detecting the signal. In one embodiment, the signal is detected using an imaging technique, such as positron emission tomography (PET), single photon emission computed tomography (SPECT), magnetic resonance imaging (MRI), radiography, tomography, fluoroscopy, nuclear medicine, optical imaging, encephalography and ultrasonography.

### BRIEF DESCRIPTION OF THE DRAWINGS

FIG. 1 illustrates the detection of synthetic Aβ oligomers in 30% human CSF by a peptide probe in accordance with the methods described herein.

FIG. 2 illustrates the selective detection of synthetic Aβ oligomers in 10% human CSF by a peptide probe in accordance with the methods described herein.

FIG. 3 is a schematic diagram of a plate-based assay that uses a biotinylated peptide probe to detect Aβ oligomers.

FIG. 4A shows the detection of synthetic Aβ oligomer in buffer by a peptide probe in accordance with the methods described herein.

FIG. 4B shows the detection of synthetic Aβ oligomer in 10% human TBS brain extract by a peptide probe in accordance with the methods described herein.

FIG. 5A shows the detection of synthetic Aβ oligomer in 10% human TBS brain extract by a peptide probe in accordance with the methods described herein.

FIG. 5B shows the detection of synthetic Aβ oligomer in 30% human TBS brain extract by a peptide probe in accordance with the methods described herein.

FIG. 6 illustrates the detection of synthetic Aβ oligomer in buffer by each of a peptide probe and two peptoid analogs as described herein.

FIG. 7 illustrates the amino acid sequences of several peptide probes useful in the methods described herein (SEQ ID NOs:1-13).

### DETAILED DESCRIPTION

### 1. Overview

Without being limited to this hypothesis, it is believed that brain trauma may result in impaired axonal transport, which in turn induces pathological co-accumulation of Amyloid Precursor Protein (APP), Aβ peptides, β-site APP-cleaving enzyme (BASE), presinilin-1 (PS-1), caspase-3 and caspase-mediated cleavage of APP (CCA) in swollen axons for up to 6 months following injury. Abnormal concentrations of these factors may lead to APP proteolysis and Aβ formation within the axonal membrane compartment.

TBI not only causes accelerated and increased Aβ deposition in plaques but also elevated brain levels of soluble Aβ40 and Aβ42. The dynamics of these amyloid beta species in the interstitial fluid of the brain directly correlate with the neurological status of the injured human brains. TBI also causes increased oxidative stress. Thus, traumatic brain injury is linked to mechanisms of AD by the fact that repetitive brain trauma accelerates brain Aβ accumulation and oxidative stress, which could synergistically promote the onset or drive the progression of AD.

Within days after traumatic brain injury, plaques form in the brain that are composed of Aβ protein, and are similar to the hallmark plaque pathology in Alzheimer's Disease (AD). However, the Aβ protein aggregates associated with TBI are not structurally identical to those associated with AD. For example, Aβ protein aggregates associated with TBI may appear “cloud like” or more diffuse as compared to Aβ protein aggregates associated with AD, which are more organized and fibrillar.

We have previously described a series of conformationally dynamic peptides based on the human amyloid beta sequence that have preferential ability to detect amyloid beta aggregates or oligomers in U.S. patent application Ser. No. 12/695,968, filed Jan. 28, 2010, the contents of which are incorporated herein by reference in their entirety. The amyloid beta sequence has been shown to be associated with the pathological effects associated with AD, and is implicated as a marker for TBI. The peptide probes, labeled at the N- and C-termini with, e.g., fluorescently active moieties, report the presence of amyloid beta aggregates by undergoing a conformational change upon binding to the aggregates, detectable due to changes in the probe's fluorescence emission profile. In the context of TBI, the peptide probes can be used to detect misfolded amyloid beta protein in biological samples, such as, for example, cerebrospinal fluid (CSF), blood or blood components, and brain tissue or extracts.

Described herein are in vitro and in vivo methods for detecting Aβ protein aggregates associated with traumatic brain injury. The in vitro methods comprise (A) contacting a physiological sample from a subject with a peptide or peptide mimic probe, wherein the probe (i) preferentially associates with the Aβ protein aggregates, (ii) undergoes a conformation shift upon association with the Aβ protein aggregates, and (iii) generates a detectable signal when the probe associates with the Aβ protein aggregates; (B) detecting any association between the probe and any Aβ protein aggregate present in the sample. The in vivo methods comprise (A) administering to a subject a peptide or peptide mimic probe that comprises a detectable label that generates a signal when the probe associates with any Aβ protein aggregates, and (B) detecting the signal. Further aspects and variations of the methods are described in more detail below.

### 2. Definitions

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any animal including humans and domesticated animals, such as cats, dogs, swine, cattle, sheep, goats, horses, rabbits, and the like. “Subject” also includes animals used in research settings, including mice and other small mammals. A typical subject may be suspected of suffering from TBI, suspected of having been exposed to conditions creating a risk for TBI, or have been exposed to such a condition, or may be desirous of determining risk or status with respect to TBI.

As used herein, “conformation” refers to the secondary or tertiary structure of a protein or peptide, for example, an alpha-helix, random coil or β-sheet secondary structure. A “conformation shift” means any change in the conformation of the protein, such as a change in the distance between the N- and C-termini (or between any other two points), folding more or less compactly, changing from predominantly one secondary structure to predominantly another secondary structure, such as from predominantly alpha helix/random coil to predominantly β-sheet, or any change in the relative amounts of different secondary structures, such as a change in the relative amounts of alpha helix/random coil and β-sheet secondary structures even without a change in the predominant secondary structure. A confirmation shift can be detected on a peptide or aggregate level. As used herein, “conformation shift” includes those shifts that can be detected by indirect means, such as through label signaling discussed below, even if more direct measures of conformation, such as CD, do not reveal a change in conformation.

The term “Aβ protein” is used herein to refer to all forms of the Aβ protein, including Aβ40 and Aβ42. “Aβ” protein also includes all naturally occurring mutants, including naturally occurring mutants known to exhibit increased tendency to form aggregates. Such mutants are known in the art, such as those disclosed in Murakami et al., J. Biol. Chem. 46:46179-46187, 2003, which is incorporated herein by reference in its entirety. Aβ is generated by cleaving the amyloid beta precursor protein (APP) at any of several sites, resulting in several forms of Aβ. Two abundant forms found in amyloid plaques are Aβ₁₋₄₀ (also referred to as Aβ40) and Aβ₁₋₄₂ (also referred to as Aβ42), which are produced by alternative carboxy-terminal truncation of APP. See, e.g., Selkoe et al., PNAS USA 85:7341-7345, 1988; Selkoe, Trends Neurosci. 16:403-409, 1993. Aβ40 and Aβ42 have identical amino acid sequences, with Aβ42 having two additional residues (Ile and Ala) at its C terminus. Although Aβ40 is more abundant, Aβ42 is the more fibrillogenic and is the major component of the two in amyloid deposits of both AD and cerebral amyloid angiopathy. See, e.g., Wurth et al., J. Mol. Biol. 319: 1279-90 (2002). Aβ42 is also the major component of aggregates associated with TBI. As noted above, all naturally occurring mutants of Aβ protein can be a target protein or serve as the basis of a reference sequence in the context of the present invention.

“Target protein” is used herein to refer to any protein whose presence is associated with TBI. In some embodiments, the protein's presence in a particular conformation or state of self-aggregation is associated with TBI; thus, “target protein” may denote a protein in a specific conformation or state of self-aggregation. In one embodiment, the target protein is Aβ protein, particularly Aβ protein aggregates that are associated with TBI. As noted above, while Aβ protein aggregates associated with AD are typically described as having fibrillar form, Aβ protein aggregates associated with TBI appear to be “cloud like” or more diffuse.

“Traumatic brain injury” (TBI) encompasses any injury to the brain. Such injuries can be caused by any sudden physical or non-physical impact to the head or body, such as from auto accidents, industrial accidents, sports injuries, explosion-generated shock or energy waves, combat or physical violence. Alternatively, the injury can be caused chemically, such as by exposure to isoflurane, anaesthesia and other chemicals associated with brain injury. The traumatic brain injury may be closed head injury, penetrating head injury, focal brain injury, diffuse brain injury, concussion, dementia pugilistica, anesthesia-related injury, isoflurane-related injury and shaken baby syndrome. Any TBI which is associated with the formation of Aβ protein aggregates may be detected using the methods of the present invention.

“Probe” refers to a peptide or peptide mimic that binds the target protein. In one embodiment, the probe binds to the target protein when the target protein has a particular conformation or is in a particular state of self-aggregation associated with TBI. In other embodiments, the probe is a conformationally dynamic peptides based on the human amyloid beta sequence, as described in U.S. patent application Ser. No. 12/695,968, filed Jan. 28, 2010, the contents of which are incorporated herein by reference in their entirety. For convenience, the peptides and peptide mimics are referred to herein as “probes” without detracting from their utility in other contexts. These probes will be discussed in more detail below.

“Native” or “naturally occurring” proteins refer to proteins recovered from a source occurring in nature. A native protein would include post-translational modifications, including, but not limited to, acetylation, carboxylation, glycosylation, phosphorylation, lipidation, acylation, and cleavage. “Protein,” “peptide” and “polypeptide” are used interchangeably.

“Peptide mimic” is also referred to as a peptidomimic or peptidomimetic or peptoid and refers to any molecule that mimics the properties of a peptide. Peptide mimics include polymeric molecules that mimic the folding and/or secondary structure of a specific peptide, as well as those that mimic the biological or chemical properties of a peptide. Peptide mimics may have an amino acid backbone and contain non-natural chemical or amino acid substitutions. Peptoids may have side chains (R-groups) on the backbone amide nitrogen, instead of the alpha carbon as in peptides. This may serve one or more of several purposes: (1) peptoids may be resistant to proteolysis; (2) since peptoid secondary structure formation does not depend on hydrogen bonding, they may exhibit enhanced thermal stability as compared to peptides, and (3) the large number of available peptoid residues allows for the production of a large variety of three-dimensional structures that may aid in assay development. Alternatively, peptide mimics may have different chemical backbones, such as β-peptides, anthranilamide oligomers, oligo (m-phenylene ethynylene), oligourea, oligopyrrolinones, azatides and N-substituted glycine oligomers. Peptide mimics may have different chemical properties, such as resistance to proteases, while retaining peptide characteristics, such as peptide folding and peptide-peptide interactions (including, for example, interactions via hydrogen bonding, etc.). Any suitable peptide mimic can be used in the present invention, and include those designed and/or constructed as described in Chongsiriwatana, N. P, et al. Proc Natl Acad Sci USA 2008, 105, (8), 2794-9; Kirshenbaum, K., et al. Current Opinion in Structural Biology 1999, 9, (4), 530-535; Lee, B. C., et al., Journal of the American Chemical Society 2005, 127, (31), 10999-11009, which are each hereby incorporated by reference in their entirety.

“Similarity” between two polypeptides is determined by comparing the amino acid sequence of one polypeptide to the sequence of a second polypeptide. An amino acid of one polypeptide is similar to the corresponding amino acid of a second polypeptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, P. (1989) EMBO J. 8:779-785. For example, amino acids belonging to one of the following groups represent conservative changes or substitutions:

-Ala, Pro, Gly, Gln, Asn, Ser, Thr:

-Cys, Ser, Tyr, Thr;

-Val, Ile, Leu, Met, Ala, Phe;

-Lys, Arg, His;

-Phe, Tyr, Trp, His; and

-Asp, Glu.

“Homology”, “homologs of”, “homologous”, “identity”, or “similarity” refers to sequence similarity between two polypeptides, with identity being a more strict comparison. Homology and identity may each be determined by comparing a position in each sequence that may be aligned for purposes of comparison. When a position in the compared sequence is occupied by the same amino acid, then the molecules are identical at that position. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. An “unrelated” or “non-homologous” sequence shares 10% or less identity, with one of the sequences described herein. Related sequences share more than 10% sequence identity, such as at least about 15% sequence identity, at least about 20% sequence identity, at least about 30% sequence identity, at least about 40% sequence identity, at least about 50% sequence identity, at least about 60% sequence identity, at least about 70% sequence identity, at least about 80% sequence identity, at least about 90% sequence identity, at least about 95% sequence identity, or at least about 99% sequence identity.

The term “percent identity” refers to sequence identity between two amino acid sequences. Identity may be determined by comparing a position in each sequence that is aligned for purposes of comparison. When an equivalent position in one compared sequences is occupied by the same amino acid in the other at the same position, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as part of the GCG sequence analysis package (University of Wisconsin, Madison, Wis.), and may be used with, e.g., default settings. ENTREZ is available through the National Center for Biotechnology Information, National Library of Medicine, NIH, Bethesda, Md.). In one embodiment, the percent identity of two sequences may be determined by the GCG program with a gap weight of 1, e.g., each amino acid gap is weighted as if it were a single amino acid mismatch between the two sequences. Other techniques for determining sequence identity are well known and described in the art.

### 3. Probes

As noted above, the peptides and peptide mimics described herein are useful, for example, for detecting target proteins, such as Aβ proteins and Aβ protein aggregates, having a specific conformation or state of self-aggregation, including Aβ protein aggregates associated with TBI. In some embodiments, the probes are conformationally dynamic peptides based on the human amyloid beta sequence, as described in U.S. patent application Ser. No. 12/695,968. The probes also may be useful in methods of screening drug candidates for treating TBI, as discussed in US 2008/0095706, the contents of which are incorporated herein by reference in their entirety.

In some embodiments, the probe comprises an amino acid sequence of the target protein that undergoes a conformational shift, such as a shift from an a-helix/random coil conformation to a β-sheet conformation. For example, amino acids 16-35 of the Aβ protein are known to comprise a β-sheet forming region. Thus, the probe may comprise amino acids 16-35, or 17-35, of the Aβ protein, or an amino acid sequence that is a variant thereof. In some embodiments, the probe comprises the amino acid sequence of a β-sheet forming region of a naturally occurring mutant of the target protein, such as a mutant known to exhibit an increased tendency to adopt a β-sheet conformation and/or to form aggregates. Examples of Aβ mutants, some of which are described in Murakami, supra, include the substitutions H6R, D7N, A21G, E22G, E22P, E22Q, E22K (“Italian”), and D23N. Other Aβ mutants include, for example, natural mutants outside the 1-42 amino acid sequence, such as the Swedish (K-2N M-1L), French (V44M), German (V44A) and London (V461 or V46G) mutants. The amino acid sequence of the peptide may be designed, therefore, from the target protein sequence, based on existing sequence and conformation information or, alternatively, may be readily determined experimentally.

In some embodiments, the peptide probe (i) consists of from 10 to 50 amino acid residues comprising an amino acid sequence that is a variant of a reference sequence consisting of an amino acid sequence of a β-sheet forming region of the target protein, (ii) is capable of adopting both a random coil/alpha-helix conformation and a a-sheet conformation, and (iii) adopts a β-sheet conformation upon binding to target protein exhibiting a β-sheet conformation or undergoes a change in conformation that generates a detectable signal upon binding to target protein. The variant sequence may comprise one or more amino acid additions, substitutions or deletions relative to the reference sequence, such that (A) the random coil/alpha-helix conformation of the variant sequence is more stable in an oxidizing environment than a probe consisting of the reference amino acid sequence and/or (B) the distance between the N-terminus and the C-terminus of the variant sequence in a random coil/alpha-helix conformation differs from the distance between the N-terminus and the C-terminus of the variant sequence in a β-sheet conformation and/or (C) the variant sequence adopts a β-sheet conformation upon binding to target protein exhibiting a β-sheet conformation more efficiently than the reference sequence and/or (D) the variant sequence adopts a less ordered conformation upon binding to target protein exhibiting a β-sheet conformation and/or (E) the β-sheet structure of the variant sequence is less thermodynamically strong than that of the reference sequence and/or (F) the variant sequence has increased stability and/or decreased reactivity than the reference sequence and/or (G) the variant sequence has an increased hydrophilicity and/or solubility in aqueous solutions than the reference sequence and/or (H) the variant sequence has an additional Aβ binding motif than the reference sequence and/or (I) the variant sequence has an enhanced ability to form aggregates. In some embodiments, the variant sequence further comprises the addition of a lysine residue at the C-terminus.

The additions, deletions and/or substitutions as compared to the amino acid sequence of the reference sequence dictate that in some embodiments, the peptide probe may have an amino acid sequence having at least 60%, at least 75%, at least 80%, at least 85%, at least 90%, at least 95%, or 100% identity to said reference sequence. In some embodiments, the peptide probe may have an amino acid sequence with one or more additional amino acids at either terminus, or at both termini, as compared to the reference sequence. Additions, substitutions, and deletions may also be made at an internal portion of the reference sequence, or both internally and terminally.

Any of the probes described herein may be endcapped at one or both of the C-terminus and the N-terminus with a small hydrophobic peptide ranging in size from about 1 to about 5 amino acids. In other embodiments, one or both of the C-terminus and N-terminus has a lysine residue, such as to facilitate labeling. Additionally or alternatively, any of the probes described herein may be modified by the substitution of a methionine residue with a residue resistant to oxidation, such as an alanine residue. Additionally or alternatively, any of the probes described herein may be modified by the substitution of at least three consecutive residues of the reference sequence with alanine residues.

Any of the probes described herein may include a dipyrene butyrate (PBA) moiety at the N-terminus and/or one extending from a lysine side chain near the C-terminus. Additionally or alternatively, any of the probes described herein may have been modified to include an amide group at the C-terminus, in place of the naturally occurring carboxyl group.

In specific embodiments, the probe may consist of two point mutations (e.g., SEQ ID NO:2); the addition of 2 d-Arginine residues (r) (e.g., SEQ ID NO:22); combinations of mutations described herein (e.g., SEQ ID NO:23); a naturally-occurring “Italian” mutant (SEQ ID NO:56); or addition of a linker and biotin (e.g., SEQ ID NO:41).

In some embodiments, the one or more amino acid additions, substitutions or deletions may introduce a salt bridge between two residues, such as between a glutamic acid residue and a histidine residue, a glutamic acid residue and an arginine residue, and/or a glutamic acid residue and a lysine residue. Further, the amino acid additions, substitutions, or deletions may introduce an Aβ binding motif into the peptide probe, such as a GXXEG motif.

As disclosed above, the variant sequence may adopt either a more- or less-ordered conformation upon binding to a target protein exhibiting a β-sheet conformation. In some embodiments, for example, the target protein is Aβ protein, and the variant sequence comprises one or more substitutions selected from the group consisting of G29H, G29R, G29K, and G33E. Additionally or alternatively, the β-sheet structure of the variant sequence may be less thermodynamically strong than that of the reference sequence. In specific embodiments, the variant sequence comprises one or more substitutions selected from the group consisting of I32S, F19S, S26D, H29D, I31D, L34D, and L34P.

In accordance with any of the foregoing embodiments, the peptide probe may be conjugated to a biotin moiety, such as through a peptide linker. In specific embodiments, the peptide linker is selected from the group consisting of a flexible linker, a helical linker, a thrombin site linker and a kinked linker. In other embodiments, the peptide probe is conjugated to a biotin moiety through a side chain of an internal lysine residue. Other appropriate peptide linkers are described in the art (see, e.g., U.S. Pat. No. 6,448,087; Wurth et al., J. Mol. Biol. 319:1279-1290 (2002); and Kim et al., J. Biol. Chem. 280:35059-35076 (2005), which are incorporated herein by reference in their entireties). In some embodiments, suitable linkers may be about 8-12 amino acids in length. In further embodiments, greater than about 75% of the amino acid residues of the linker are selected from serine, glycine, and alanine residues.

For example, biotinylation can be achieved through a helical linker such as EAAAK at the C-terminus, as illustrated by AD310 (SEQ ID NO:38). In general, a helical linker includes residues that form alpha helixes, such as alanine residues. Alternatively, biotinylation can be achieved through a side chain on a lysine residue, including an internal or terminal lysine residue, as illustrated by AD313 (SEQ ID NO:39). Alternatively, biotinylation can be achieved through a flexible linker (such as GSSGSSK) at the C-terminus, as illustrated by AD314 (SEQ ID NO:40). In general, a flexible linker includes one or more glycine and/or serine residues, or other residues that can freely rotate about their phi and psi angles. Alternatively, biotinylation can be achieved through a thrombin site linker (such as a linker comprising LVPRGS, such as GLVPRGSGK) at the at the C-terminus, as illustrated by AD317 (SEQ ID NO:41). Alternatively, biotinylation can be achieved through a kinked linker (such as PSGSPK) at the at the C-terminus, as illustrated by AD321 (SEQ ID NO:42). In general, kinked linkers comprise one or more proline residues, or other residues that have fixed phi and psi angles that rigidly project the biotin moiety away from the peptide probe's protein-binding motif

Additionally or alternatively, the variant sequence may have an increased hydrophilicity and/or solubility in aqueous solutions than the reference sequence. In specific embodiments, the variant sequence comprises one or more amino acid additions or substitutions that introduce a glutamic acid residue and/or a d-arginine residue. Additionally or alternatively, the variant sequence may be conjugated to a hydrophilic moiety, such as a soluble polyethylene glycol moiety.

In some embodiments, the variant sequence comprises the substitution of at least one residue with a glutamic acid residue. In some embodiments, the variant sequence comprises the substitution of at least one residue with a histidine residue. In some embodiments, the variant sequence comprises one or more substitutions selected from the group consisting of an isoleucine residue with a serine residue; glutamic acid residue with either a proline residue, a glycine residue, a glutamine residue or a lysine residue; a phenylalanine residue with a serine residue; a leucine residue with a proline residue; an alanine residue with a glycine residue; and an aspartic acid residue with an asparagine residue.

The probe may comprise a minimum number of contiguous amino acids of the target protein, such as at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 41, at least about 42, at least about 43, at least about 44, at least about 45, at least about 46, or at least about 50 contiguous amino acids of the target protein sequence, or any range between these numbers, such as about 10 to about 25 contiguous amino acids of the target protein sequence.

The probe may comprise a maximum number of contiguous amino acids of the target protein, such as up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20, up to about 21, up to about 22, up to about 23, up to about 24, up to about 25, up to about 30, or up to about 35 contiguous amino acids of the target protein sequence, or any range between these numbers, such as about 10 to about 25 contiguous amino acids of the target protein sequence.

The reference sequence may comprise a minimum number of contiguous amino acids of the target protein, such as at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 41, at least about 42, at least about 43, at least about 44, at least about 45, at least about 46, or at least about 50 contiguous amino acids of the target protein sequence, or any range between these numbers, such as about 10 to about 25 contiguous amino acids of the target protein sequence.

The reference sequence may comprise a maximum number of contiguous amino acids of the target protein, such as up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20, up to about 21, up to about 22, up to about 23, up to about 24, up to about 25, up to about 30, or up to about 35 contiguous amino acids of the target protein sequence, or any range between these numbers, such as about 10 to about 25 contiguous amino acids of the target protein sequence.

The probes themselves may comprise at least about 5 amino acids, and may include up to about 300 to about 400 amino acids, or more, or any size in between, such as about 10 amino acids to about 50 amino acids in length. In some embodiments, the peptides consist of about 5 to about 100, about 10 to about 50, about 10 to about 25, about 15 to about 25, or about 20 to about 25 amino acids. In further embodiments, the peptides comprise from about 17 to about 34 amino acids, including about 20 amino acids, about 21 amino acids, about 22 amino acids, about 23 amino acids, about 24 amino acids, or about 25 amino acids. Peptides of different lengths may exhibit different degrees of interaction and binding to the target protein, and suitable lengths can be selected by the skilled artisan guided by the teachings herein.

In some embodiments, the probes are selected from SEQ ID NOs: 1-56. In some specific embodiments, the probes are selected from the group consisting of SEQ ID NOs: 2, 22, 23, 56, and 41. Probes described in US 2008/0095706 for targeting Aβ protein, and probes designed in accordance with U.S. patent application Ser. No. 12/695,968, may be used as described herein. The contents of these applications are incorporated herein by reference in their entirety.

Exemplary peptide probes designed in accordance with the principles described above are set forth in Table 1 below. As shown by shading in the sequences, most of the peptide sequences are based on amino acids 16-35 of the Aβ peptide (WT; SEQ ID NO:1), which is a β-sheet forming region of the Aβ peptide (others are based on longer portions of the Aβ peptide), with an added C-terminal lysine residue to facilitate labeling. The category (or categories) of the sequence variants are indicated in the table (e.g., modified to improve stability, provide a salt bridge, increase solubility, facilitate alpha-helix formation, destabilize β-sheet structure, add an Aβ binding motif, etc.). Also illustrated are options for peptide probe labeling, including different label sites and label pairs. Unless indicated otherwise, all peptides were labeled with two pyrene labels, one on the N-terminal amine, and the other on a side chain of a C-terminal lysine residue. Additionally, unless indicated otherwise, all constructs contain a C-terminal amide in place of the carboxyl group.

The following abbreviations are used in the table:

“PBA” =pyrene butyric acid

“r”=d-Arginine

“Dabcyl”=4-(4-dimethylaminophenyl) diazenylbenzoic acid

“EDANS”=5-(2′-aminoethyl)aminonaphthalene-1-sulfonic acid

“FAM”=5(6)carboxyfluorescein

“Dansyl”=5-dimethylaminonaphthalene-1-sulfonyl

TABLE 1
Peptide Probes

| SEQ | | | | |
|---|---|---|---|---|
| ID | | | | |
| NO: | Category | Name | Modification | Sequence |
| 1 | Wildtype | WT | Aβ protein residues 16-35, with added C-Terminal Lys | 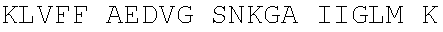 |
| 6 | Stability | AD250 | M35A to replace oxidizable methionine residue | 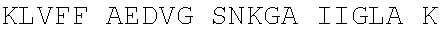 |
| 2 | Salt Bridge | P22 | Salt bridge at G29H and G33E, also induce alpha- helix, and increase solubility | 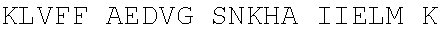 |
| 14 | | P22 v.1 | Salt bridge at G29R and G33E | 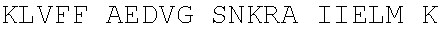 |
| 15 | | P22 v.2 | Salt bridge at G29K and G33E | 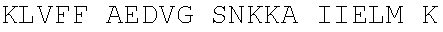 |
| 3 | Salt Bridge + Alpha Helix | P38 | Salt bridge at G29H and G33E; Ala substitutions to increase alpha- helicity | 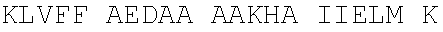 |
| 4 | | P45 | Salt bridge at G29H and G33E; Ala additions to increase alpha-helicity | 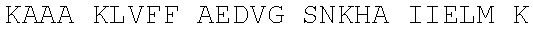 |
| 16 | Salt Bridge + Aβ Binding Motif | P77 | Salt bridge; Additional Aβ binding motif (GxxEG; SEQ ID NO: 25); extended N-terminus | 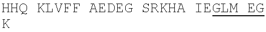 |
| 17 | | P59 | 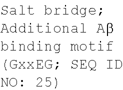 | 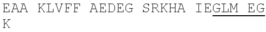 |
| 19 | Based on Naturally Occurring Mutants | Italian | P22, with E22K point mutation | 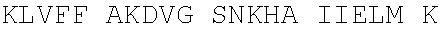 |
| 20 | | Dutch | P22, with E22Q point mutation | 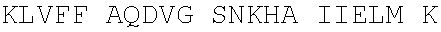 |
| 21 | | Arctic | P22, with E22G point mutation | 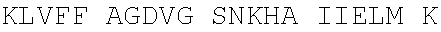 |
| 22 | Solubility | AD272 | WT, with 2 C-terminal dArg residues, and alternalte label site | 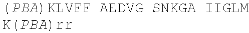 |
| 23 | | AD316 | P22, with 2 C-terminal dArg residues, and alternalte label site | 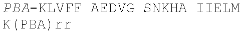 |
| 24 | | AD305 | P22, with 2 N-terminal dArg residues, 2 C-terminal E residues and alternalte label site | 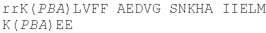 |
| 1 | | AD274 | WT, with PEG10 at C-terminus | 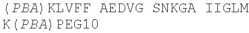 |
| 26 | | AD271 | P45, with two dArg residues at C-terminus | 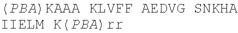 |
| 27 | Induce Alpha- Helix + Solubility | AD273 | WT, with addition of Ala stretch (for alpha- helix formation) and dArg residues (for solubility) | 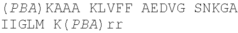 |
| 28 | Reduce Stability of B-sheet | AD323 | P22, with point mutations H29D and I31D | 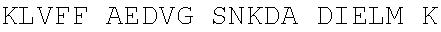 |
| 29 | | AD325 | P22, with point mutation S26D | 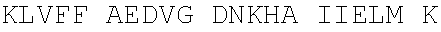 |
| 30 | | AD330 | P22, with point mutation I31D | 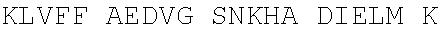 |
| 31 | | AD329 | P22, with point mutation L34D | 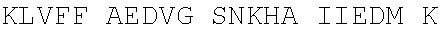 |
| 32 | | AD328 | P22, with point mutation H29D | 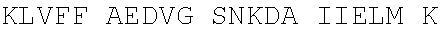 |
| 33 | | AD327 | P22, with point mutation S26D, I31D | 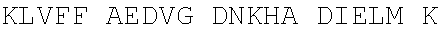 |
| 34 | | GM6 | P22, with point mutations F19S, L34P | 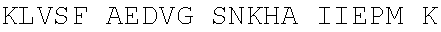 |
| 35 | | GM6 var.1 | P22, with point mutation F19S | 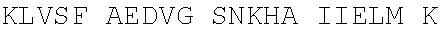 |
| 5, 18 | | I32S | Wildtype, with I32S point mutation | 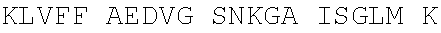 |
| 36 | Label (PBA) Site | AD266 | WT, with label on side chain of N-terminal Lys | 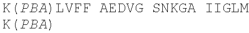 |
| 37 | | AD268 | WT, with label on side chain of near N-terminal Lys; addition of solubilizing dArg and E residues | 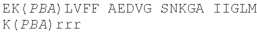 |
| 38 | Biotin | AD310 | P22, biotin labeled with helical linker at C-terminus | 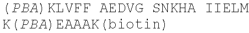 |
| 39 | | AD313 | P22, biotin labeled at side chain of internal Lys | 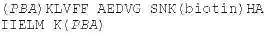 |
| 40 | | AD314 | P22, biotin labeled with flexible linker at C-terminus | 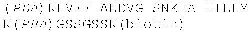 |
| 41 | | AD317 | P22, biotin labeled with thrombin site linker, at C-terminus | 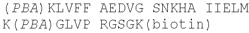 |
| 42 | | AD321 | P22, biotin labeled with “kinked” linker at C-terminus | 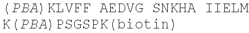 |
| 2, 43 | Label/ Quencher Pairs | AD326 | P22, with pyrene and Dabcyl quencher | 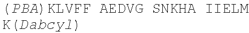 |
| 44 | | AD309 | WT, with EDANS and Dabcyl quencher and solubilizing E residue | 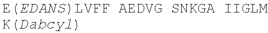 |
| 45 | | AD306 | Wildtype Aβ residues 5-42, with EDANS and Dabcyl quencher and solubilizing E residue | 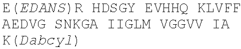 |
| 46 | | AD303 | Wildtype Aβ residues 3-35, with EDANS and Dabcyl quencher and solubilizing E residue | 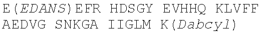 |
| 47 | | AD302 | P59, with EDANS and Dabcyl quencher and solubilizing E residue | 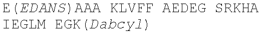 |
| 48 | | AD301 | P77, with EDANS and Dabcyl quencher and solubilizing E residue | 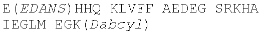 |
| 49 | | AD300 | P22 with EDANS and Dabcyl quencher and solubilizing E residue | 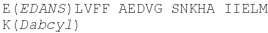 |
| 50 | FRET Pairs | AD295 | P22, with Dansyl and Trp | 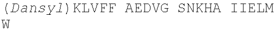 |
| 51 | | AD294 | WT, with FAM and EDANS and solubilizing E residue | 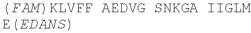 |
| 52 | | AD293 | P22,with FAM and EDANS and solubilizing E residue | 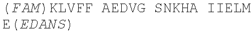 |
| 53 | | AD292 | Aβ residues 3-35, with FAM and EDANS and solubilizing E residue | 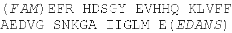 |
| 54 | | AD291 | P77, with FAM and EDANS and solubilizing E residue | 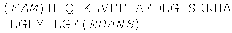 |
| 55 | | AD290 | P59, with FAM and EDANS, additional Ala, and solubilizing E residue | 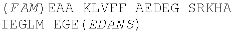 |

The probe may alternatively be a peptide mimic (“peptoid”) of any of the peptide probes described herein. In some embodiments, the probe is a peptide mimic that has a natural peptide backbone but has non-natural amino acids or chemical moieties. In other embodiments, the probe is a peptide mimic that has a non-peptide backbone and comprises a chemical backbone, such as a polymeric backbone. In some embodiments, a peptide mimic exhibits increased stability over the corresponding peptide.

Additional probes may be designed and tested for use in the present methods. Briefly, peptides and peptide mimics may be computationally designed to closely match hydrophobic topology and intramolecular pair contacts to wild type Aβ peptide (SEQ ID NO:1) and/or a probe with the desired characteristics as described above. Algorithms for designing such peptides and peptide mimics are known in the art. See, e.g., Mobley, D. L., et al., Structure 2009, 17, (4), 489-98; Fennell, C. J., et al., J Phys Chem B 2009; Voelz, V. A., et al., PLoS Comput Biol 2009, 5, (2), e1000281.; Shell, M. S., et al., Biophys J 2009, 96, (3), 917-24; Mobley, D. L., et al., J Chem Theory Comput 2007, 3, (4), 1231-1235; Wu, G. A., et al., Structure 2008, 16, (8), 1257-66; Chorny, I., et al., J Phys Chem B 2005, 109, (50), 24056-60.

The probes described herein selectively associate with target protein and undergo a conformation shift upon association with target protein. For example, in some embodiments, the probes described herein bind to Aβ protein aggregates associated with TBI and undergo a conformation shift upon such binding. As noted above, the conformation shift may comprise a change in the distance between the N- and C-termini of the probe (or between any other two points), folding more or less compactly, changing from predominantly one secondary structure to predominantly another secondary structure, or any change in the relative amounts of different secondary structures. As noted above, “conformation shift” includes those shifts that can be detected by indirect means, such as through label signaling discussed below, even if more direct measures of conformation, such as CD, do not reveal a change in conformation.

In some embodiments, the probe undergoes a conformation change similar to that of the target protein. For example, in some embodiments, the probes are capable of adopting both a primarily random coil/alpha-helix conformation and a primarily β-sheet conformation, and adopt a primarily β-sheet conformation upon binding to target protein exhibiting a primarily β-sheet conformation. In some embodiments the probe is provided in a primarily α-helix/random coil conformation, and undergoes a conformation shift to a primarily β-sheet conformation upon contact, binding, association and/or interaction with target protein in a primarily β-sheet conformation. In other embodiments, the probe shifts conformation by becoming more condensed, more diffuse, or adopting any different configuration. In some embodiments, the probe more closely adopts the conformation of the Aβ protein aggregates.

For in vitro uses, the probe may be provided in a solution, such as an aqueous solution with a pH of between about 4 and about 10, such as between about 5 and about 8, with an ionic strength of between about 0.01 and about 0.5 (when typically prepared with a chloride salt, such as sodium chloride or potassium chloride). The solution may also comprise a water-miscible organic material (e.g., trifluoroethanol, hexafluoro-2-propanal (HFIP) or acetonitrile (ACN)) in amounts between about 30% to about 100% by volume, such as between about 45% to about 60%. The solvent may be prepared with a suitable buffering system such as acetate/acetic acid, Tris, or phosphate. For in vivo uses, the probe may be provided in any physiologically acceptable solution. For example, the probe may be prepared as a trifluoracetic salt and resuspended in an organic solvent, such as 100% HFIP or 50% ACN.

### 4. Labels

As noted above, the probes disclosed herein may comprise one or more detectable labels. For example, the probe may be coupled or fused, either covalently or non-covalently, to a label. In some embodiments, the labels are selected to permit detection of a specific conformation of the probe, such as the conformation adopted when the probe associates with Aβ protein aggregates associated with TBI. In this scenario, the label may emit a first signal (or no signal) when the probe is in a first, unassociated conformation (such as a primarily random coil/alpha-helix conformation or less organized or less dense form) and a second signal, or no signal (i.e., the probe is quenched) when the probe undergoes a conformational shift upon association with target protein (such as a primarily β-sheet conformation or more organized or more dense form). The first signal and second signal may differ in one or more attributes, such as intensity, wavelength, etc. In embodiments where the signal includes emission of light, the first signal and second signal may differ in excitation wavelength and/or emission wavelength. The signal generated when the probe undergoes a conformation shift may result from interactions between labels bound to the same probe and/or may result from interactions between labels bound to different probes.

In some embodiments, a peptide probe may be labeled with a detectable label at the N-terminus, the C-terminus, both termini, or at one or more positions that generate a signal when the peptide adopts a β-sheet conformation or undergoes a conformation change upon binding to target protein. The peptide probe may be labeled with two or more labels, wherein the distance between two or more labels on the peptide probe when the peptide probe is bound to target protein is different than the distance when the peptide probe is not bound to target protein. The peptide probe may additionally or alternatively be labeled with a detectable label pair selected from an excimer pair, a FRET pair and a fluorophore/quencher pair. When the peptide probe is labeled with an excimer pair, such as a pyrene pair, it may emit an excimer signal when the peptide probe exhibits a β-sheet conformation. When the peptide probe is labeled with a FRET pair, such as DACIA-I/NBD, Marina Blue/NBD, Dansyl/Trp, and EDANS/FAM, it may emit a fluorescence resonance transfer (FRET) signal when the peptide probe exhibits a β-sheet conformation. When the peptide probe is labeled with a fluorophore/quencher pair, such as pyrene/Dabcyl, EDANS/Dabcyl and FAM/Dabcyl, the fluorophore signal may be quenched when the peptide probe exhibits a β-sheet conformation.

In accordance with any of the foregoing, a detectable label may be conjugated to a side chain of a terminal lysine residue of the peptide probe, and/or to a side chain of an internal lysine residue of the peptide probe.

In some embodiments, the labels and label sites are selected such that the labels do or do not interact based on the conformation of the probe, for example, such that the labels do not interact when the probe is in its unassociated conformation and do interact when the probe undergoes a conformation shift upon association with target protein, to generate a detectable signal (including quenching), or vice versa. This may be accomplished by selecting label sites that are further apart or closer together depending on the associated state of the probe, e.g., depending on whether the probe has undergone a conformation shift upon association with target protein. In some embodiments, the magnitude of the signal associated with the associated probe is directly correlated to the amount of target protein detected. Thus, the methods of the present invention permit detection and quantification of target protein.

For example, excimer, FRET or fluorophore/quencher label pairs may be used to permit detection of a specific conformation of the probe, such as the conformation adopted when the probe associates with Aβ protein aggregates associated with TBI. In these embodiments, the probe is labeled at separate sites with a first label and a second label, each being complementary members of an excimer, FRET or fluorophore/quencher pair.

For example, excimer-forming labels may emit their monomeric signals when the probe is in its unassociated state, and may emit their excimer signal when the probe undergoes a conformation shift that brings the labels in closer physical proximity, upon association with the target protein. Similarly, FRET labels may emit their FRET signal when the probe undergoes a conformation shift that brings the labels in closer physical proximity. On the other hand, fluorophore/quencher label pairs may emit the fluorophore signal when the probe is in its unassociated state, and that signal may be quenched when the probe undergoes a conformation shift that brings the labels in closer physical proximity. As noted above, the labels may be sited such that the opposite change in signal occurs when the probe undergoes a conformation shift upon association with the target protein.

In some embodiments, the probe is endcapped (at one or both ends of the peptide) with a detectable label. In some embodiments, the probe comprises a detectable label at or near its C-terminus, N-terminus, or both. For example, the probe may comprises a detectable label at its C-terminus, N-terminus, or both, or at other sites anywhere that generate a signal when the probe undergoes a conformation shift upon association with Aβ protein aggregate associated with TBI. Thus, for example, the label sites may be selected from (i) the N-terminus and the C-terminus; (ii) the N-terminus and a separate site other than the C-terminus; (iii) the C-terminus and a separate site other than the N-terminus; and (iv) two sites other than the N-terminus and the C-terminus.

In one embodiment, pyrene moieties are present at or near each terminus of the probe and the ratio of the pyrene monomer signal to the pyrene excimer signal is dependent upon the conformation of the probe, because the pyrene moieties may be separated by different distances depending on the conformation of the peptide, such as the pyrenes being in close physical proximity in the β-sheet conformation and further apart in the random coil/alpha-helix conformation. For example, the peptide adopts a β-sheet conformation in water, with the pyrene moieties in relatively close proximity (about 10 ↑ between the centers of the N- and C-terminal pyrene rings). In contrast, the peptide adopts an alpha-helix conformation in 40% trifluoroethanol (TFE), with the pyrene moieties further apart (about 20 ↑ between the centers of the N- and C-terminal pyrene rings). Thus, for example, the monomer signal may predominate when the probe is in its unassociated state, and the excimer signal may predominate when the probe undergoes a conformation shift upon association with target protein (or the excimer signal may increase without necessarily becoming predominant). Thus, the ratio of the pyrene monomer signal to the pyrene excimer signal may be measured. Pyrene moieties present at other sites on the probe also may be useful in this context, as long as excimer formation is conformation dependent.

The formation of excimers may be detected by a change in optical properties. Such changes is may be measured by known fluorimetric techniques, including UV, IR, CD, NMR, or fluorescence, among numerous others, depending upon the fluorophore label. The magnitude of these changes in optical properties is directly related to the amount of probe that has adopted the conformation associated with the signal, and so is directly related to the amount of target protein or structure present.

While these embodiments have been described in detail with regard to excimer pairs, those skilled in the art will understand that similar considerations apply to FRET and fluorophore/quencher pairs.

Moreover, while these embodiments have been described with reference to the use two labels per peptide probe, it should be understood that multiple labels could be used. For example, one or more labels could be present at each labeling site, or multiple labels could be present, each at different labeling sites on the probe. In these embodiments, the labels may generate independent signals, or may be related as excimer pairs, FRET pairs, signal/quencher, etc. For example, one site might comprise one, two, three, four or more pyrene moieties and another site might comprise a corresponding quencher.

Exemplary labels include fluorescent agents (e.g., fluorophores, fluorescent proteins, fluorescent semiconductor nanocrystals), phosphorescent agents, chemiluminescent agents, chromogenic agents, quenching agents, dyes, radionuclides, metal ions, metal sols, ligands (e.g., biotin, streptavidin haptens, and the like), enzymes (e.g., beta-galactosidase, horseradish peroxidase, glucose oxidase, alkaline phosphatase, and the like), enzyme substrates, enzyme cofactors (e.g., NADPH), enzyme inhibitors, scintillation agents, inhibitors, magnetic particles, oligonucleotides, and other moieties known in the art. Where the label is a fluorophore, one or more characteristics of the fluorophore may be used to assess the associated state of the labeled probe. For example, the excitation wavelength of the fluorophore may differ based on whether the labeled probe is in its unassociated conformation, or in the conformation adopted upon association with target protein. In some embodiments, the emission wavelength, intensity, or polarization of fluorescence may vary based on the associated state of the labeled probe.

As used herein, a “fluorophore” is a chemical group that may be excited by light to emit fluorescence or phosphorescence. A “quencher” is an agent that is capable of quenching a fluorescent signal from a fluorescent donor. A first fluorophore may emit a fluorescent signal that excites a second fluorophore. A first fluorophore may emit a signal that is quenched by a second fluorophore. The probes disclosed herein may undergo fluorescence resonance energy transfer (FRET).

Fluorophores and quenchers may include the following agents (or fluorophores and quenchers sold under the following tradenames): 1,5 IAEDANS; 1,8-ANS; umbelliferone (e.g., 4-Methylumbelliferone); acradimum esters, 5-carboxy-2,7-dichlorofluorescein; 5-Carboxyfluorescein (5-FAM); 5-Carboxytetramethylrhodamine (5-TAMRA) ; 5-FAM (5-Carboxyfluorescein); 5-HAT (Hydroxy Tryptamine) ; 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680 ™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL ; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate ; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue ; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8 ; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS ; Dihydorhodamine 123 (DHR); DiI (DiIC18(3)); Dinitrophenol; DiO (iOC18(3)); DiR; DiR (DiIC18(7)); DNP; Dopamine; DsRed; DTAF; DY-630-NHS; DY-635-NHS; EBFP; ECFP; EGFP; ELF 97; EDANS; Eosin; Erythrosin; Erythrosin ITC; Ethidium Bromide; Ethidium homodimer -1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); a fluorescent protein (e.g., GFP (S65T); GFP red shifted (rsGFP); GFP wild type, non-UV excitation (wtGFP); GFP wild type, UV excitation (wtGFP); and GFPuv); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; luminol, Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin ; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin E8G; Oregon Green; Oregon Green 488-X; Oregon Green™; Oregon Green™488; Oregon Green™500; Oregon Green™514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110 ; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT ; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11 ; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

As noted above, in some embodiments, the label comprises a pyrene moiety. As used herein, a pyrene moiety includes pyrene, which comprises four fused benzene rings or a derivative of pyrene. By pyrene derivative is meant a molecule comprising the four fused benzene rings of pyrene, wherein one or more of the pyrene carbon atoms is substituted or conjugated to a further moiety. Exemplary pyrene derivatives include alkylated pyrenes, wherein one or more of the pyrene carbon atoms is substituted with a linear or branched, substituted or unsubstituted, alkyl, alkenyl, alkynyl or acyl group, such as a C₁-C₂₀, linear or branched, substituted or unsubstituted alkyl, alkenyl, alkynyl or acyl group, where the group may be substituted with, for example, a moiety including an O, N or S atom (e.g., carbonyl, amine, sulfhydryl) or with a halogen. In some embodiments the pyrene derivative includes one or more free carboxyl groups and/or one or more free amine groups, each of which may be directly attached to a pyrene carbon atom or attached to any position on a linear or branched, substituted or unsubstituted, alkyl, alkenyl, alkynyl or acyl group as described above, such as being attached at a carbon atom that is separated from a pyrene carbon by 1 or more, such as 1 to 3, 1 to 5, or more, atoms. In some embodiments, the pyrene is substituted with one or more acetic acid moieties and/or one or more ethylamine moieties. In some embodiments, the pyrene derivative is substituted with a single methyl, ethyl, propyl or butyl group. In some embodiments, the pyrene is substituted with a short chain fatty acid, such as pyrene butyrate. In another embodiment, the pyrene is conjugated to albumin, transferring or an Fc fragment of an antibody. In some embodiments, the substituent is attached to pyrene through a carbon-carbon linkage, amino group, peptide bond, ether, thioether, disulfide, or an ester linkage. In other embodiments, the pyrene derivative is PEGylated pyrene, i.e., pyrene conjugated to polyethylene glycol (PEG). Such pyrene derivatives may exhibit a longer circulating half-life in vivo. In other embodiments, the pyrene derivative is pyrene conjugated to albumin.

In some embodiments, the label comprises a fluorescent protein which is incorporated into a probe as part of a fusion protein. Fluorescent proteins may include green fluorescent proteins (e.g., GFP, eGFP, AcGFP, TurboGFP, Emerald, Azami Green, and ZsGreen), blue fluorescent proteins (e.g., EBFP, Sapphire, and T-Sapphire), cyan fluorescent proteins (e.g., ECFP, mCFP, Cerulean, CyPet, AmCyan1, and Midoriishi Cyan), yellow fluorescent proteins (e.g., EYFP, Topaz, Venus, mCitrine, YPet, PhiYFP, ZsYellow1, and mBanana), and orange and red fluorescent proteins (e.g., Kusabira Orange, mOrange, dTomato, dTomato-Tandem, DsRed, DsRed2, DsRed-Express (T1), DsREd-Monomer, mTangerine, mStrawberry, AsRed2, mRFP1, JRed, mCherry, HcRed1, mRaspberry, HcRed-Tandem, mPlum and AQ143). Other fluorescent proteins are described in the art (Tsien, R. Y., Annual. Rev. Biochem. 67:509-544 (1998); and Lippincott-Schwartz et al., Science 300:87-91 (2003)).

As noted above, the probes may be comprised in fusion proteins that also include a fluorescent protein coupled at the N-terminus or C-terminus of the probe. The fluorescent protein may be coupled via a peptide linker as described in the art (U.S. Pat. No. 6,448,087; Wurth et al., J. Mol. Biol. 319:1279-1290 (2002); and Kim et al., J. Biol. Chem. 280:35059-35076 (2005), which are incorporated herein by reference in their entireties). In some embodiments, suitable linkers may be about 8-12 amino acids in length. In further embodiments, greater than about 75% of the amino acid residues of the linker are selected from serine, glycine, and alanine residues.

In some embodiments, the label comprises an oligonucleotide. For example, the probes may be coupled to an oligonucleotide tag which may be detected by known methods in the art (e.g., amplification assays such as PCR, TMA, b-DNA, NASBA, and the like).

In embodiments comprising in vivo detection or imaging, labels useful for in vivo imaging can be used. For example, labels useful for magnetic resonance imaging, such as fluorine -18 can be used, as can chemiluminescent labels. In another embodiment, the probe is labeled with a radioactive label. For example, the label may provide positron emission of a sufficient energy to be detected by machines employed for this purpose. One example of such an entity comprises oxygen-15 (an isotope of oxygen that decays by positron emission) or other radionuclide. Another example is carbon-11. Probes labeled with such labels can be administered to a patient, permitted to localize at sites containing Aβ protein aggregates associated with TBI, and the patient can be imaged (scanned) to detect localized probe, and thus identify sites of localized target protein. The imaging techniques that may be used include, inter alia, magnetic resonance imaging (MRI), radiography, tomography, fluoroscopy, nuclear medicine, optical imaging, encephalography and ultrasonography.

### 5. Methods

As discussed above, the present invention provides both in vitro and in vivo methods for the detection of Aβ protein aggregates associated with TBI.

The in vitro methods may be useful for the detection of Aβ protein aggregates associated with TBI in a physiological sample from a subject by contacting the sample with a probe that preferentially associates with the Aβ protein aggregate and undergoes a conformation shift upon the association.

Aβ protein aggregates associated with TBI may be localized in the brain, and/or may be present at other sites. Thus, in accordance with the methods described herein, a “physiological sample” is any sample from a subject that may be tested for Aβ protein aggregates, and includes, inter alia, brain tissue, cerebrospinal fluid, whole blood, serum, plasma, eye tissue, vascular tissue, lung tissue, kidney tissue, heart tissue and liver tissue.

The physiological sample may be prepared for use in the present methods in any manner compatible with the present methods, for example homogenization, cell disruption, dilution, clarification, etc. Care may be taken to not denature the proteins in the physiological sample so that the target protein retains its original conformation. The physiological sample may optionally be further processed prior to the addition of the probe using conventional techniques, such as sonication.

Detection of the association of the probe and Aβ protein aggregate can be effected by several different methods. For example, the probe-Aβ protein aggregate complexes can be separated from other constituents of the reaction mixture, such as unbound probe and/or unbound Aβ protein, and then the complexes can detected by detecting the detectable label on the probe present in the complex, or by detecting the signal emitted by the probe when it undergoes a conformation shift upon association with the target protein (the “target-associated signal”). Separation can be accomplished using any method known in the art.

In some embodiments, the probe-Aβ protein aggregate complex is separated using size exclusion chromatography (SEC). SEC retains smaller molecules using pores or openings in the capture media (also termed stationary phase) such that the smaller molecules migrate more slowly through capture media while the larger molecules pass through more quickly. These pores or openings are of defined size and can be selected to differentiate between the probe-Aβ protein aggregate complex and unbound probe and/or unbound Aβ protein. In accordance with these methodologies, the complex will elute before unbound probe. Detection of the detectable label on the probe (or of the target-associated signal) in earlier fraction(s) is correlated with the presence of probe-Aβ protein aggregate complex, which in turn is correlated with Aβ protein aggregates associated with TBI in the test sample.

An alternative embodiment uses affinity chromatography to retain the probe-Aβ protein aggregate complex on the capture media. This approach utilizes a capture media, such as a solid phase, that comprises an affinity molecule that binds to the probe-Aβ protein aggregate complex. The affinity molecule can be selected to specifically bind the Aβ protein aggregate, the probe, the complex, or a label conjugated to any component of the probe-Aβ protein aggregate complex. In some embodiments, the affinity molecule specifically binds the Aβ protein aggregate or a label conjugated to it such that the Aβ protein aggregate is retained on the capture media. Once unbound constituents (including any unbound probe) are washed off, the bound material can be eluted, typically using an elution buffer and the eluant can be analyzed. Detection of the detectable label on the probe (or of the target-associated signal) in the eluant is correlated with the presence probe-Aβ protein aggregate complex in the eluant, which in turn is correlated with Aβ protein aggregates associated with TBI in the test sample.

An alternative method for detecting target protein in a test sample, wherein the target protein exhibits a β-sheet conformation associated with TBI, comprises (i) contacting the sample with any peptide probe described herein to form a test mixture; and (ii) detecting any binding between the peptide probe and any target protein present.

In some embodiments, step (ii) comprises detecting any signal generated by the fluorescent label of peptide probe exhibiting a β-sheet conformation or undergoing a conformational change upon binding to a target protein. In some embodiments, step (ii) comprises detecting complexes comprising the peptide probe and target protein by detecting any signal generated by any detectable label (such as a fluorescent label) present in the complexes. In some embodiments, the complexes are insoluble complexes (such as amyloid beta fibrils) and step (ii) comprises detecting any signal generated by any detectable label (such as a fluorescent label) present in the insoluble complexes. In some embodiments, the complexes are soluble complexes (such as amyloid beta oligomers) and step (ii) comprises detecting any signal generated by any detectable label (such as a fluorescent label) present in the soluble complexes. In some embodiments, the method further comprises, prior to step (ii), separating the complexes from the test mixture by a process comprising centrifugation, size exclusion chromatography, or affinity chromatography.

A further method for detecting target protein associated with TBI, may comprise (A) contacting the sample with a peptide probe that is a peptide or peptide mimic that (i) consists of from 10 to 50 amino acid residues comprising an amino acid sequence that is a variant of a reference sequence consisting of an amino acid sequence of a β-sheet forming region of the target protein, (ii) is capable of adopting both a random coil/alpha-helix conformation and a β-sheet conformation, and (iii) adopts a less ordered conformation upon binding to target protein; and (B) detecting any association between said probe and any target protein present in the sample. In some embodiments, the peptide probe may be labeled with a detectable label at the N-terminus, the C-terminus, both termini, or at one or more positions that generate a signal when the peptide undergoes a conformation change upon binding to target protein. In specific embodiments, the peptide probe may be labeled with an excimer pair and step (ii) comprises detecting any increased self signal or decreased excimer signal. In other embodiments, the peptide probe may be labeled with a FRET pair and step (ii) comprises detecting any increased non-FRET fluorophore signal or decreased FRET signal. In other embodiments, the peptide probe is labeled with a fluorophore/quencher pair and step (ii) comprises detecting any increased fluorophore signal.

As noted above, in some embodiments, association or binding between the probe and Aβ protein aggregate is detected by detecting a signal generated by the probe, such as a signal generated when the probe undergoes a conformation shift upon association or binding with a Aβ protein aggregate associated with TBI. These embodiments may be effected either with or without separation of probe-Aβ protein aggregate complex from the reaction mixture (such as described above). In these embodiments, the probe may be labeled with an excimer-forming label, such as pyrene, with FRET labels, or with fluorophore/quencher labels, as described above, and a signal is generated (or quenched) when the probe undergoes as conformation shift, such as may occur upon association, contact, interaction or binding with Aβ protein aggregates associated with TBI.

Further, there is provided an in vivo method for detecting target protein associated with TBI in a subject, comprising (A) administering to the subject any peptide probe as described herein, wherein the probe is labeled with a detectable label that generates a signal when the probe binds to target protein and (B) detecting the signal. In some embodiments, the signal is detected using an imaging technique, such as positron emission tomography (PET), single photon emission computed tomography (SPECT), magnetic resonance imaging (MRI), radiography, tomography, fluoroscopy, nuclear medicine, optical imaging, encephalography and ultrasonography.

In other embodiments, there is provided a method of treating a subject suffering from or at risk of developing TBI, comprising administering to the subject any peptide probe described herein. In some embodiments, the probe is conjugated to an additional therapeutic agent against said TBI.

In embodiments related to in vivo detection, a subject is administered a peptide or peptide mimic probe that is labeled with a detectable label that generates a signal when the probe associates with any Aβ protein aggregates, the probe is permitted to localize at sites of Aβ protein aggregates, and the signal is detected, such as by scanning or imaging. Further details on in vivo methodologies are provided, for example, in US 2008/0095706, the contents of which are incorporated herein by reference in their entirety. Labeled probes can be administered by any suitable means that will permit localization at sites of target protein, such as by direct injection, intranasally or orally. As noted above, Aβ protein aggregates associated with TBI may be localized in the brain, and/or may be present at other sites. Thus, in accordance with the methods described herein suitable sites for localization and imaging include at least the brain, CSF region, blood, serum, plasma, eyes, lungs, kidneys, hearts and liver. In some embodiments, labeled probes can be injected into a patient and the association of the probe to the target protein monitored externally, such as by positron emission tomography (PET), single photon emission computed tomography (SPECT), magnetic resonance imaging (MRI), radiography, tomography, fluoroscopy, nuclear medicine, optical imaging, encephalography and ultrasonography.

### 6. Kits

Also provided are kits comprising the probes described herein. The kits may be prepared for practicing the methods described herein. Typically, the kits include at least one component or a packaged combination of components useful for practicing a method. By “packaged combination” it is meant that the kits provide a single package that contains a combination of one or more components, such as probes, buffers, instructions for use, and the like. A kit containing a single container is included within the definition of “packaged combination.” The kits may include some or all of the components necessary to practice a method disclosed herein. Typically, the kits include at least one probe in at least one container. The kits may include multiple probes which may be the same or different, such as probes comprising different sequences and/or different labels, in one or more containers. Multiple probes may be present in a single container or in separate containers, each containing a single probe.

### EXAMPLES

### Example 1

### Peptide Probes

Probes for the detection of Aβ aggregates were designed in accordance with the principles described herein. As illustrated in Table 1 and FIG. 8, these peptide sequences are based on amino acids 17-35 of the Aβ peptide, which is a β-sheet forming region of the Aβ peptide. The reference sequence (WT; SEQ ID NO:1) corresponds to the wildtype sequence, with a terminal lysine residue added to facilitate pyrene labeling. These peptides have been shown to bind preferentially to Aβ protein and undergo a conformation shift to generate a signal, as described in U.S. patent application Ser. No. 12/695,968. Specific exemplary peptide probes are o described below in Table 2. These probes include modifications that make them more soluble in aqueous solution compared to the reference Aβ peptide sequence. These probes include a dipyrene butyrate (PBA) moiety at the N-terminus and one extending from a lysine side chain near the C-terminus. Additionally, they have been modified to include an amide group at the C-terminus, in place of the naturally occurring carboxyl group.

TABLE 2

| SEQ | |
|---|---|
| ID | Sequence |
| 1 | PBA-KLVFF AEDVG SNKGA IIGLM K(PBA)-NH₂ |
| 2 | PBA-KLVFF AEDVG SNKHA IIELM K(PBA)-NH₂ |
| 22 | PBA-KLVFF AEDVG SNKGA IIGLM K(PBA)rr-NH₂ |
| 23 | PBA-KLVFF AEDVG SNKHA IIELM K(PBA)rr-NH₂ |
| 56 | PBA-KLVFF AKDVG SNKGA IIGLM K(PBA)-NH₂ |
| 41 | PBA-KLVFF AEDVG SNKHA IIELM K(PBA)GLVPR |
| | GSGK(biotin)-NH₂ |

The ability of other probes selected and/or designed in accordance with the description herein to preferentially associate with Aβ aggregates associated with TBI can be assessed and confirmed by methods described in US 2008/0095706 and U.S. patent application Ser. No. 12/695,968. For example, a bead-based oligomer binding assay, in which probe-oligomer complexes are immuno-precipitated with monoclonal 6E10 antibody and protein G-agarose can be used.

The 6E10 antibody is specific to the N-terminus of Aβ 42 peptide (1-10aa), which corresponds to an epitope not found in the probe. Therefore, the antibody will only bind to full length Aβ protein which may be present, not to the probe. To perform this assay, the TBI sample/probe reaction mixture is equilibrated to ensure binding of 6E10 monoclonal antibody to oligomers. After brief incubation, the antibody is precipitated with protein G-agarose beads, and washed to remove all unbound proteins. The bead-associated proteins are eluted and characterized with SDS PAGE and Western blot. The level of probe binding is estimated by comparison to reference standards to confirm the presence of TBI-associated Aβ aggregates in the sample.

### Example 2

### Detection of Synthetic Aβ Aggregates in Media

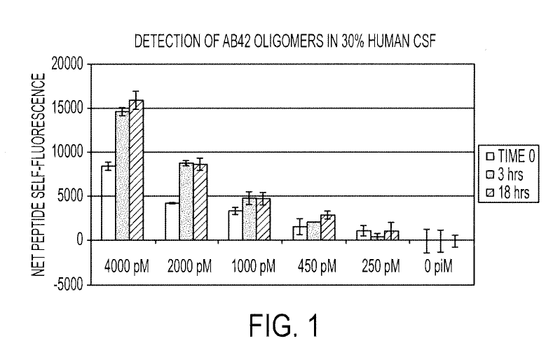

70 nM of the peptide probe of SEQ ID NO:2 is incubated with 4000, 2000, 1000, 450, 250 and 0 pM synthetic Aβ42 oligomer (in triplicate) in a solution consisting of 10 mM Hepes (pH 7.0), 0.0074% Tween20 and 30% (v/v) normal human CSF (Bioreclamations, Inc.) for 0, 3, and 18 hours at room temperature in a final volume of 200 μL in a microtiter plate. The plate is then analyzed using a Tecan safire² fluorescence plate reader. For each sample, the net self-fluorescence response (fluorescence emission from 370-385 nm) is determined by subtraction of the self-fluorescence response of the control (0 pM) from the fluorescence response of the experimental sample. As shown in FIG. 1, as little as 450 pM of Aβ42 oligomer is statistically distinguishable from the control reaction (t-test).

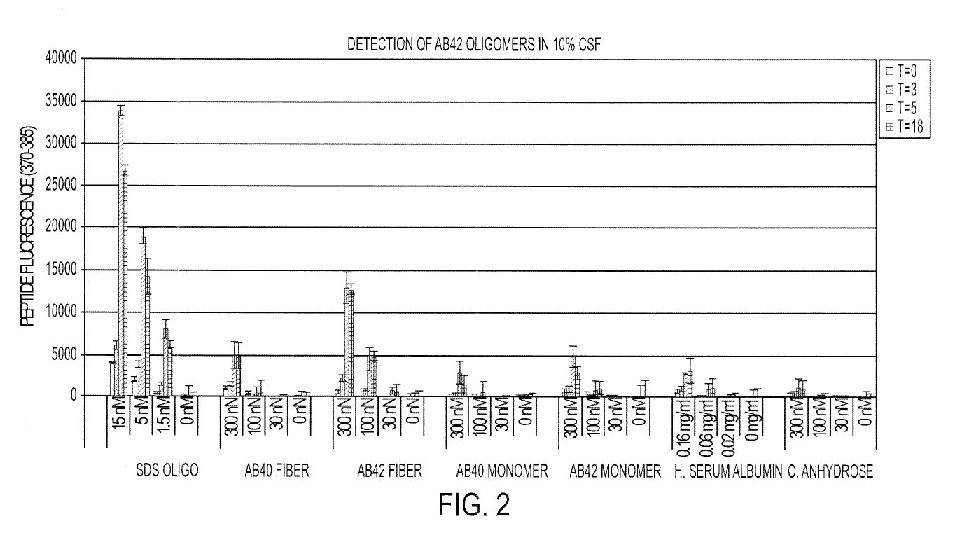

The specificity of the probe is confirmed as follows. 70 nM of the peptide probe of SEQ ID NO:22 is incubated with several potential substrates in a solution consisting of 10 mM Hepes (pH 7.0), 0.0074% Tween20 and 10% (v/v) normal human CSF. As shown in FIG. 2, this probe is reactive with amyloid beta fibers and highly reactive with Aβ oligomers. There is a strong and dose-dependent response of the peptide probe to Aβ42 oligomer. There also is a significant response to Aβ42 fiber down to at least 100 nN. In contrast, there is little or no peptide fluorescence response to Aβ40 fiber, Aβ40 and Aβ42 monomer, human serum albumin (except at the highest dose, 0.16 mg/mL, which is the approximate physiological concentration), or carbonic anhydrase. Since fibers are easily removed from the reaction by a centrifugation step, specificity for amyloid beta oligomers is easily obtained. Thus, these data illustrate that amyloid beta aggregates are specifically and selectively detectable in a physiologically relevant media.

### Example 3

### Detection of Synthetic Aβ Aggregates in Brain Extract

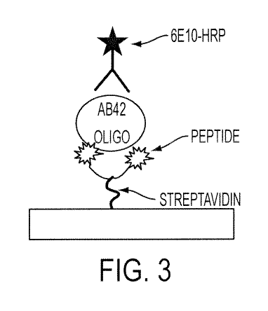

A plate-based ELISA-like assay was developed in which the above-described peptide probes may be used to capture and detect target amyloid beta protein (such as oligomers). FIG. 3 shows a schematic diagram of an exemplary plate-based assay. Streptavidin-coated 96-well plates are prepared, followed by introduction of biotinylated peptide. Sample is then added to the wells and allowed to incubate, such as for two hours at room temperature. Any target amyloid beta protein in the sample will be capture by the immobilized peptide probe. The plate is then washed, such as using a low salt buffer to eliminate potential interfering factors (e.g. endogenous proteins, lipids, and other debris). The remaining amyloid beta aggregate-peptide probe complex is bound to a reporter antibody that is specific for the N-terminus of the amyloid beta sequence (6E10-HRP), and detected by addition of 3,3′,5,5′-tetramethylbenzidine (TMB).

Such an assay is used to confirm the ability of peptide probes to detect Aβ42 oligomers in the presence of either buffer or 10-30% brain extract, using samples spiked with synthetic Aβ42 oligomers.

Soluble brain extracts are prepared according to a known method. For example 5 mL tris-buffered saline (PH 7.4) is added per gram of frozen brain tissue, and then homogenized with a dounce homogenizer (25 strokes). The material is then centrifuged at 21,900× g for 30 minutes at 4 C. The resulting TBS supernatant (soluble extract) is used at 10-30% (v/v) final concentration in the following assays.

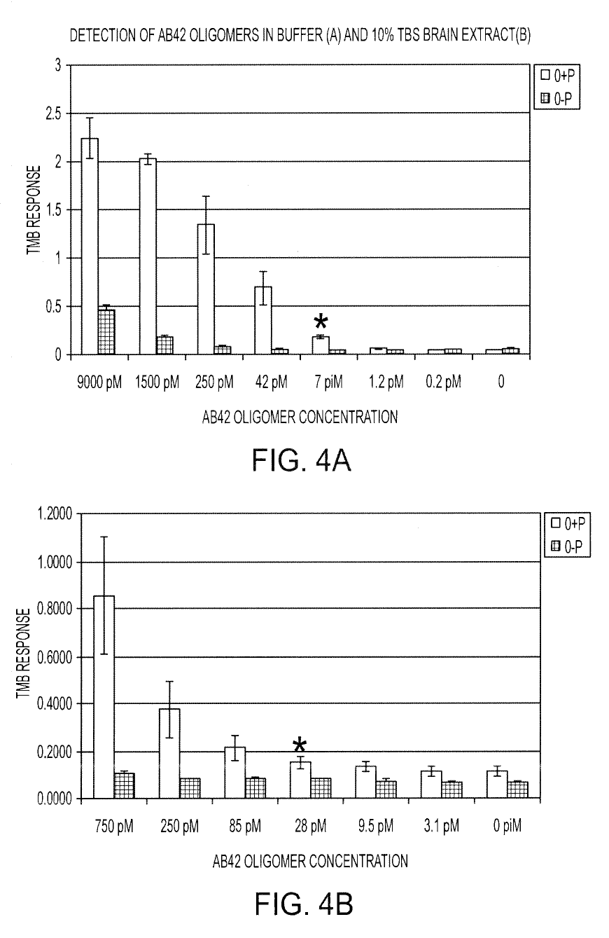

FIG. 4A and FIG. 4B illustrate the results in buffer and 10% soluble mouse brain extract using SEQ ID NO:41 as a peptide probe. FIG. 4A shows a synthetic Aβ42 oligomer titration in a buffer system. The white bars indicate control reactions in which peptide are not added. Black bars show the complete reaction in which 9000, 1500, 250, 42, 7, 1.2, 0.2, or 0 pM Aβ42 oligomer is added (in triplicate) to wells containing bound peptide probe of SEQ ID NO:41. The data show that as little as 7 pM of amyloid beta aggregate can be detected in the assay (t-test).

FIG. 4B shows a synthetic Aβ42 oligomer titration in the presence of 10% human TBS brain extract. The white bars indicate control reactions in which peptide are not added. Black bars show the complete reaction in which 10% human TBS brain extract containing 750, 250, 85, 28, 9.5, 3.1, or 0 pM Aβ42 oligomer is added (in triplicate) to wells containing bound peptide probe of SEQ ID NO:41. The data show that as little as 28 pM of amyloid beta aggregate can be detected in the assay (t-test).

The oligomer dose response shows that the sensitivity of the plate-based assay is in the low pM range both in buffer and in the presence of 10% soluble mouse brain extract. Moreover, this response shows specificity with respect to peptide (scrambled biotinylated peptide does not interact with amyloid beta oligomers), and with respect to substrate (Table 3). That is, there is little to no TMB response observed when biotinylated peptide probe is challenged with amyloid beta monomers (both Aβ42 and Aβ40), or human serum albumin. Nor is there a TMB response when a scrambled sequence variant of SEQ ID NO:41 is used instead of SEQ ID NO:41. Table 3 also shows that similar sensitivity is observed with a different type of synthetic Aβ42 oligomer.

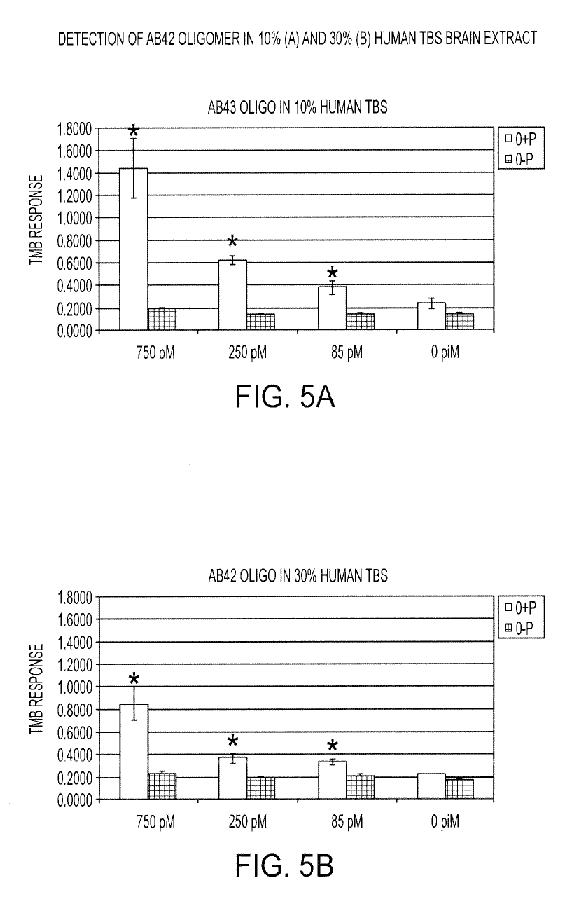

FIG. 5A and FIG. 5B illustrate the results in 10% (A) and 30% (B) human brain TBS extract using SEQ ID NO:41 as a peptide probe. FIG. 5A shows a synthetic Aβ42 oligomer titration in the presence of 10% human TBS brain extract. The white bars indicate control reactions in which peptide is not added. Black bars show the complete reaction in which 10% human TBS brain extract containing 750, 250, 85 or 0 pM Aβ42 oligomer is added (in triplicate) to wells containing bound peptide probe of SEQ ID NO:41.

FIG. 5B shows a synthetic Aβ42 oligomer titration in the presence of 30% human TBS brain extract. The white bars indicate control reactions in which peptide are not added. Black bars show the complete reaction in which 30% human TBS brain extract containing 750, 250, 85, 28, 9.5, 3.1, or 0 pM Aβ42 oligomer is added (in triplicate) to wells containing bound peptide probe of SEQ ID NO:41. The data show that, although there is some suppression of overall signal with increasing brain extract content, amyloid beta aggregate is detectable in a milieu of 30% TBS brain extract down to at least 85 pM.

Similar experiments have been performed in the presence of normal human CSF to show that the plate-based assay is compatible with this physiological media. Notably, similar sensitivity and specificity characteristics of the peptide probes were observed.

TABLE 3
Specificity of Peptide Probe in Plate Assay

| | |
|---|---|
| Human | Mouse |

Limit of detection

| | | |
|---|---|---|
| Aβ42 Oligo | ~28 pM | ~28-85 pM |
| (Type A) | | |
| Aβ42 Oligo | 8.5 pM | ~8.5 pM |
| (Type B) | | |
| Aβ40 Dimer | Not tested | 850 pM |
| Aβ42 Monomer | Not detected | Not detected |
| | (up to 15 nM) | (up to 15 nM) |
| Aβ40 Monomer | Not detected | Not detected |
| | (up to 15 nM) | (up to 15 nM) |
| has | Not detected | Not detected |
| | (up to 0.15 mg/mL) | |
| Scrambled | Not Tested | Not detected |
| Peptide | | (up to 750 pM |
| | | AB42 Oligo) |

### Example 4

### Detection of Synthetic Aβ Aggregates Using Peptoids

Two peptoid analogs of the peptide probe of SEQ ID NO:2 were prepared and tested for their ability to interact with amyloid beta aggregates. Modeling studies suggest that these structures should form a compact structure analogous to the beta sheet structure observed in the peptide probes under aqueous conditions. Additionally, the distance between the two pyrene moieties is comparable to what is observed for peptide probes (˜10-15 Å).

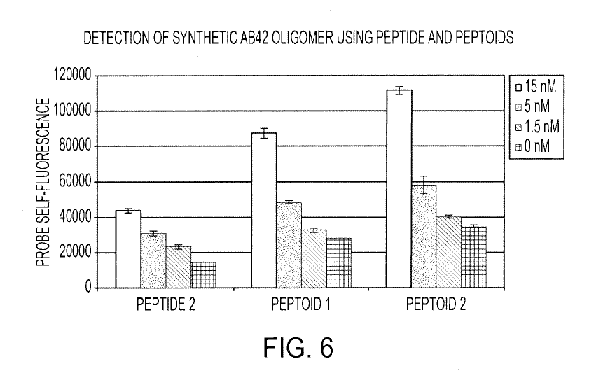

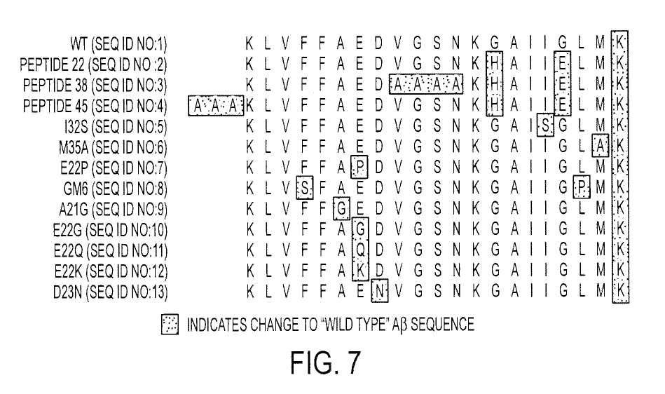

These two peptoids are used in an assay as shown in FIG. 6. 70 nM of each of the two peptoid probes 1 or 2 is incubated with 15, 5, 1.5 or 0 nM synthetic Aβ42 oligomer (in triplicate). The reactions are performed in 10 mM Hepes (pH 7.0) at room temperature in a final volume of 200 μL in a microtiter plate. The plate is then analyzed using a Tecan safire² fluorescence plate reader. For each sample, the self-fluorescence response (fluorescence emission from 370-385 nm) of the peptoid is plotted as a function of amyloid beta aggregate concentration. The amyloid beta aggregate dose response of the three probe structures is comparable.

A variant of these peptoids in which biotin is appended can be synthesized for use in assays, such as the plate assay described above.

### Example 5

### Detection of Aβ Aggregates in TBI Mice

Controlled cortical impact (CCI) surgery: TBI is induced in mice using a CCI-injury device. The CCI-injury device was designed and built at Georgetown University, and consists of a microprocessor-controlled pneumatic impactor with a 3.5 mm diameter tip (Chomy et al.). Mice are anaesthetized with isoflurane (induction at 4% and maintenance at 1.5%) evaporated in a gas mixture containing 70% N₂O and 30% O₂ and administered through a nose mask. Depth of anesthesia is assessed by monitoring respiration rate and pedal withdrawal reflexes. The mouse is placed on a heated pad, and core body temperature is maintained at 37° C. The head is mounted in a stereotaxic frame, and the surgical site is clipped and cleaned with Nolvasan scrubs. A 10-mm midline incision is made over the skull, the skin and fascia reflected, and a 4-mm craniotomy is made on the central aspect of the left parietal bone. The impounder tip of the injury device is then extended to its full stroke distance (44 mm), positioned to the surface of the exposed dura, and reset to impact the cortical surface. Injury is induced by an impactor velocity of 6 m/s and deformation depth of 2 mm. After injury, the incision is closed with interrupted 6-0 silk sutures, anesthesia terminated, and the animal is placed into a heated cage to maintain normal core temperature for 45 minutes post-injury. All animals are monitored carefully for at least 4 hours after surgery. Surgeries for individual studies are performed by the same model expert within as short a timeframe as feasible to minimize experimental variation, with sham and TBI groups randomly intermingled.

Euthanasia and Tissue collection: Twenty four hours following surgery, euthanasia is performed using CO₂ inhalation according to GUACUC guidelines. Brains for immunohistochemistry are drop fixed in 10% formalin in PBS for 24 hours, followed by 24 hours in 20% sucrose in PBS, then 24 hours in 30% sucrose in PBS. Six mm of brain, including 3 mm rostral to 3 mm caudal to the injury epicenter, is frozen and cut on a cryostat to create 20 μm coronal sections through the injury site. Brains for biochemistry are immediately dissected into ipislateral and contralateral cortex and snap frozen on dry ice.

96 well plate format assay: TBI samples obtained above, along with reference standards containing synthetic Aβ42 oligomers at concentrations ranging from 1 pM to 1 μM are incubated with 0.1-4 μM of peptide probe, in a preferred embodiment biotinylated Pronucleon peptide, labeled with excimer (pyrene) or FRET label pairs in a solution with 10 mM Hepes (pH 7.0). Reactions are incubated in the dark at room temperature (21-25° C.).

Fluorescence measurements are taken at time 0, 3 hours and 18 hours. Pyrene excimer or FRET fluorescence of TBI sample and Aβ42 oligomer-containing reference samples is compared to a buffer control.

By comparing the fluorescent signals of the TBI samples to those of the reference standards, the presence and amount of TBI-associated Aβ aggregates can be determined.

Specificity of the assay is validated by testing against Aβ monomer, Aβ fibrils, Alzheimer relevant proteins such as tau, other peptides involved in neurodegenerative diseases such as α-synuclein, a panel of abundant serum proteins such as BSA and protein irrelevant to Alzheimer's disease.

What is claimed is:

1. A method for detecting Aβ protein aggregates associated with traumatic brain injury in a physiological sample from a subject, comprising:
(A) contacting the sample with a peptide or peptide mimic probe, wherein said probe (i) preferentially associates with said Aβ protein aggregates, (ii) undergoes a conformation shift upon association with said Aβ protein aggregates, and (iii) generates a detectable signal when said probe associates with said Aβ protein aggregates; and
(B) detecting any association between said probe and any Aβ protein aggregate present in said sample.

2. The method of claim 1, wherein said probe is labeled with a detectable label that generates a signal when said probe associates with said Aβ protein aggregates.

3. The method of claim 2, wherein said probe is labeled at separate sites with a first label and a second label, generating a signal when said probe undergoes said conformation shift upon association with said Aβ protein aggregates.

4. The method of claim 3, wherein said sites of said first label and second label are selected from (i) the N-terminus and the C-terminus; (ii) the N-terminus and a separate position other than the C-terminus; (iii) the C-terminus and a separate position other than the N-terminus; and (iv) two positions other than the N-terminus and the C-terminus.

5. The method of claim 3, wherein said first and second labels are excimer-forming labels.

6. The method of claim 5, wherein said first and second labels comprise pyrene.

7. The method of claim 3, wherein said first label comprises one member of a fluorescent resonance energy transfer (FRET) pair and said second label comprises the other member of said FRET pair.

8. The method of claim 7, wherein said FRET pair is selected from DACIA-NBD, Marina Blue/NBD, EDNAS/Fam (fluorescein), Dabcyl/EDANS and Dabcyl-FAM.

9. The method of claim 3, wherein said first and second labels constitute a fluorophore/quencher pair.

10. The method of claim 3, wherein said conformation shift is selected from the group consisting of (a) adopting a conformation upon association with said Aβ protein aggregate that increases the physical proximity of said first and second labels; and (b) adopting a conformation upon association with said Aβ protein aggregate that decreases the physical proximity of said first and second labels.

11. The method of claim 1, wherein said physiological sample is selected from brain tissue, cerebrospinal fluid, whole blood, serum, plasma, eye tissue, vascular tissue, lung tissue, kidney tissue, heart tissue and liver tissue.

12. The method of claim 1, wherein said probe is a peptide probe.

13. The method of claim 12, wherein said peptide probe consists of from 10 to 50 amino acid residues corresponding to a β-sheet forming region of Aβ protein, wherein the amino acid sequence of said probe is at least 60%, at least 75%, at least 80%, at least 85%, at least 90%, at least 95%, or 100% identical to said corresponding region of Aβ protein.

14. The method of claim 1, wherein said probe is a peptide mimic.

15. The method of claim 14, wherein said probe is a peptide mimic of a peptide consisting of from 10 to 50 amino acid residues corresponding to a β-sheet forming region of Aβ protein

16. The method of claim 1, wherein the traumatic brain injury is due to physical or chemical trauma.

17. The method of claim 16, wherein the traumatic brain injury is selected from the group consisting of closed head injury, penetrating head injury, focal brain injury, diffuse brain injury, concussion, dementia pugilistica, anesthesia-related injury, isoflurane-related injury and shaken baby syndrome.

18. An in vivo method for detecting Aβ protein aggregates associated with traumatic brain injury, comprising:
(A) administering to the patient a peptide or peptide mimic probe, wherein said probe (i) preferentially associates with said Aβ protein aggregate, (ii) undergoes a conformation shift upon association with said Aβ protein aggregate, and (iii) is labeled with a detectable label that generates a signal when said probe associates with said Aβ protein aggregates; and
(B) detecting said signal.

19. The method of claim 18, wherein said signal is detected using an imaging technique.

20. The method of claim 19, wherein said imaging technique is selected from the group consisting of positron emission tomography (PET), single photon emission computed tomography (SPECT), magnetic resonance imaging (MRI), radiography, tomography, fluoroscopy, nuclear medicine, optical imaging, encephalography and ultrasonography.
